(19) United States
(12) Patent Application Publication
(10) Pub. No.: US 2018/0264219 A1
(43) Pub. Date: Sep. 20, 2018

(54) INTERACTIVE SPACER FOR RESPIRATORY DEVICE

(71) Applicant: THE RESEARCH INSTITUTE AT NATIONWIDE CHILDREN'S HOSPITAL, Columbus, OH (US)

(72) Inventors: Evan KILGORE, Denver, CO (US); Amber Marie PATTERSON, Findlay, OH (US); Jeremy PATTERSON, Columbus, OH (US); Melissa ROBINSON, Denver, CO (US); Robert V. STROUSE, Columbus, OH (US)

(21) Appl. No.: 15/753,476

(22) PCT Filed: Aug. 29, 2016
(86) PCT No.: PCT/US16/49233

§ 371 Date: Feb. 19, 2018

Related U.S. Application Data
(60) Provisional application No. 62/211,098, filed on Aug. 28, 2015.
(60) Provisional application No. 62/379,904, filed on Aug. 26, 2016.

(51) Int. Cl. A61M 16/06; A61M 11/00

(52) U.S. Cl. CPC A61M 16/0605; A61M 11/00; A61M 2205/13; A61M 2205/18; A61M 2210/0625; A61M 2205/581; A61M 2205/583; A61M 2210/0618; A61M 2205/43

(57) ABSTRACT

A respiratory device for dispensing medication provided comprising a mask for covering a patient's mouth and/or nose, a medication dispenser for housing medication to be received by a patient upon activation of a button via depression of a lever coupled to a spacer body of the an interactive spacer. The button located on the dispenser. The interactive spacer eccentrically located and spacing the mask from the medication dispenser, the spacer comprises a power supply end an interactive controller for providing at least one of sound and light upon activation of first switch located on the device.

### CROSS REFERENCES TO RELATED APPLICATIONS

The following application claims priority under 33 U.S.C. § 119(c) to co-pending U.S. Provisional Patent Application Ser. No. 62/211,098 filed Aug. 28, 2015 entitled INTERACTIVE SPACER FOR RESPIRATORY DEVICE and to co-pending U.S. Provisional Patent Application Ser. No. 62/379,904 filed Aug. 26, 2016 entitled INTERACTIVE SPACER FOR RESPIRATORY DEVICE. The above-identified applications are incorporated herein by reference in their entireties for all purposes.

### TECHNICAL FIELD

The present disclosure relates generally to an interactive spacer for a respiratory device, and more specifically, an interactive spacer that notifies and/or trains the operator and/or their assistant while using the device.

### BACKGROUND

Respiratory devices relate generally to drug delivery apparatuses, typically used by children. A more common name for such a device is an inhaler, which often provides medication relating to respiratory problems, although it could provide medication relating to other ailments in addition to, or alternatively to respiratory issues.

In some instances, the respiratory devices include an incentive attachment, such as a toy or training device. Such examples can be found in European Patent Publication Nos. EP 0 667 168 to Aradigm Corporation and EP 2 747 814 to Koninlijke Philips N. V., and U.S. Pat. No. 4,534,343 to Nowacki et al., U.S. Pat. No. 6,578,571 to Watt, and U.S. Patent Publication No. 2014/0257126 to Vink et a. All of the above-identified patents and applications being incorporated herein by reference in their entireties for alt purposes.

### SUMMARY

One example embodiment of the present disclosure includes a respiratory device for dispensing medication is provided comprising a mask for covering a patient's mouth and/or nose, a medication dispenser for housing medication to be received by a patient upon activation of a button by a lever located on a spacer body of an interactive spacer. The button is located on the dispenser. The interactive spacer is eccentrically located and spaces the mask from the medication dispenser, the spacer comprises a power supply and an interactive controller for providing at least one of sound and light upon activation of a first switch located on the device.

One example embodiment of the present disclosure includes a method of dispensing medication from a respiratory device, the method comprises activating a first switch by identifying that a mask is covering at least one of a nose and mouth of a patient. The mask is coupled to a medication dispenser by an interactive spacer, wherein the interactive spacer is eccentrically located and spaces said mask from said medication dispenser. The first switch is in electrical communication with a controller located in the interactive spacer. The method further comprises identifying a depression of a lever to activate a button located on the dispenser, wherein the lever is coupled to a spacer body of the interactive spacer. The button in electrical communication with the controller. The method additionally comprises responsive to activating the first switch and identifying the depression of the lever dispensing the medication from the medication dispenser through the interactive spacer to the mask and providing at least one of sound and light as provided by the controller in electrical communication with at least one of a speaker and an illumination assembly.

One example embodiment of the present disclosure includes a respiratory device for dispensing medication is provided comprising a mask for covering a patient's mouth and/or nose, a medication dispenser for housing medication to be received by a patient upon activation of a button by a lever located on a spacer body of an interactive spacer. The button is located on the dispenser. The interactive spacer is eccentrically located and spaces the mask from the medication dispenser. The interactive spacer comprises a passage for the medication along a first axis and the mask includes an opening in fluid communication with said passage along a second axis transverse to said first axis. The spacer comprises a power supply and interactive controller for providing at least one of sound and light upon activation of a first switch located within said mask. Wherein the first switch is activated by contact of a patient's face upon making a sufficient seal with said mask during use.

### BRIEF DESCRIPTION OF THE DRAWINGS

The foregoing and other features and advantages of the present disclosure ii become apparent to one skilled in the art to which the present disclosure relates upon consideration of the following description of the invention with reference to the accompanying drawings, wherein like reference numerals, unless otherwise described refer to like parts throughout the drawings and in which:

FIG. 1A is an illustration of an interactive spacer for a respiratory device in accordance with one example embodiment of the present disclosure being used by a helper on a patient;

FIG. 1 is an illustration of an interactive spacer for a respiratory device in accordance with one example embodiment of the present disclosure being used by a patient;

FIG. 2 is a left side elevation view of the interactive spacer device of FIG. 1;

FIG. 3 is a right side elevation view of the interactive spacer device of FIG. 1;

FIG. 4 is a front partial elevation view of an interactive spacer and a mask in accordance with the embodiments illustrated in FIGS. 1-3;

FIG. 5 is a schematic illustrating the electronic circuitry of the interactive spacer for a respiratory device in accordance with another example embodiment of the present disclosure;

FIG. 6 illustrates use of the interactive spacer for a respiratory device in accordance with the embodiment of FIG. 1;

FIG. 7 is a bottom elevation view of the interactive spacer device of FIG. 1;

FIG. 8 is a right side perspective view of the interactive spacer device of FIG. 1;

FIG. 9 is a left side perspective view of the interactive spacer device of FIG. 1;

FIG. 10 is an illustration of an interactive spacer for a respiratory device in a relaxed position in accordance with another example embodiment of the present disclosure;

FIG. 11 is an illustration of an interactive spacer for a respiratory device in an engaged position in accordance with another example embodiment of the present disclosure;

FIG. 12 is a loft side perspective view of the interactive spacer device of FIG. 10;

FIG. 13 is a left side perspective view of the interactive spacer device of FIG. 11;

FIG. 14 is a top plan view of the interactive spacer device in accordance with an example embodiment of the present disclosure;

FIG. 15 is a front elevation view of the interactive spacer device in accordance with an example embodiment of the present disclosure;

FIG. 16 is a rear elevation view of the interactive spacer device in accordance with an example embodiment of the present disclosure;

FIG. 17 is a right side elevation view of the interactive spacer device in accordance with an example embodiment of the present disclosure;

FIG. 18 is a left side elevation view of the interactive spacer device in accordance with an example embodiment of the present disclosure;

FIG. 19 is a right side perspective view of the interactive spacer device in accordance with an example embodiment of the present disclosure;

FIG. 20 is a left side perspective view of the interactive spacer device in accordance with an example embodiment of the present disclosure;

FIG. 21 is a perspective view of a lever of an interactive spacer device in accordance with an example embodiment of the present disclosure;

FIG. 22 is a side elevation view of the lever of FIG. 21;

FIG. 23 is a rear elevation view of an elbow of an interactive spacer device in accordance with an example embodiment of the present disclosure;

FIG. 24 is a front elevation view of the elbow of FIG. 23;

FIG. 25 is a front perspective view of the elbow of FIG. 23;

FIG. 26 is a cross section view the elbow taken along lines 26-26 of FIG. 25;

FIG. 27 is a side elevation view of the elbow of FIG. 23;

FIG. 28 is a rear elevation view of an elbow and mask of an interactive spacer device in accordance with an example embodiment of the present disclosure;

FIG. 29 is a front elevation of an interactive spacer device in accordance with an example embodiment of the present disclosure;

FIG. 30 is a left side perspective exploded assembly view of an interactive spacer device in accordance with an example embodiment of the present disclosure;

FIG. 31 is a right side perspective exploded assembly view of an interactive spacer device in accordance with an example embodiment of the present disclosure;

FIG. 32 is a right side elevation exploded assembly view of an interactive spacer device in accordance with an example embodiment of the present disclosure;

FIG. 33 is a right side elevation view of an interactive spacer device, wherein a spacer body of the interactive spacer device is depicted in phantom in accordance with an example embodiment of the present disclosure; and

FIG. 34 is a right side elevation view of an interactive spacer device, wherein a spacer body of the interactive spacer device is depicted in phantom in accordance with another example embodiment of the present disclosure.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present disclosure.

The apparatus and method components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

### DETAILED DESCRIPTION

Referring now to the figures wherein like numbered features shown therein refer to like elements throughout unless otherwise noted. The present disclosure relates generally to an interactive spacer for a respiratory device, and more specifically, an interactive spacer that notifies and/or trains the operator and/or their assistant while using the device.

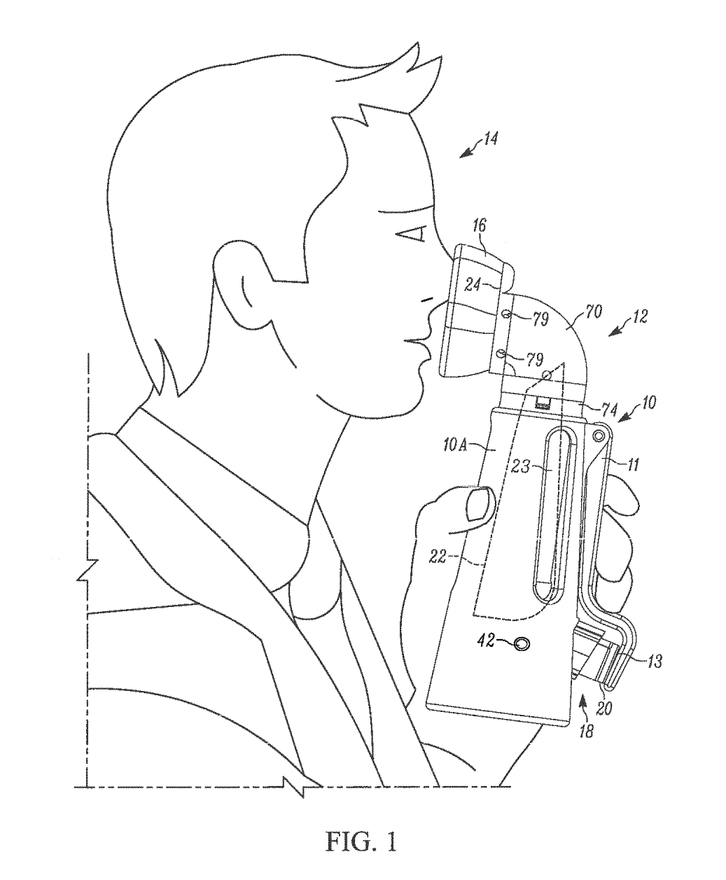

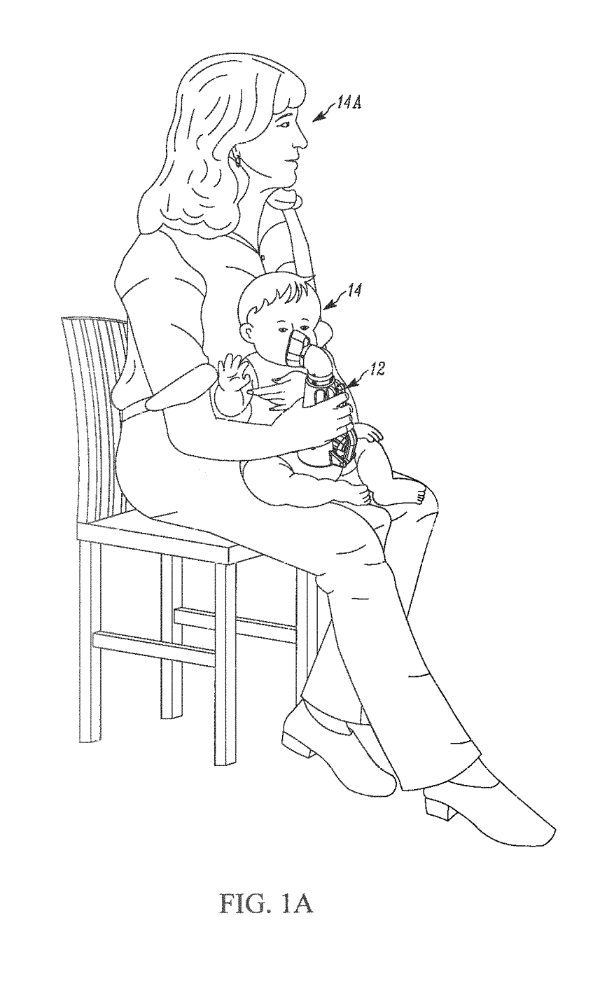

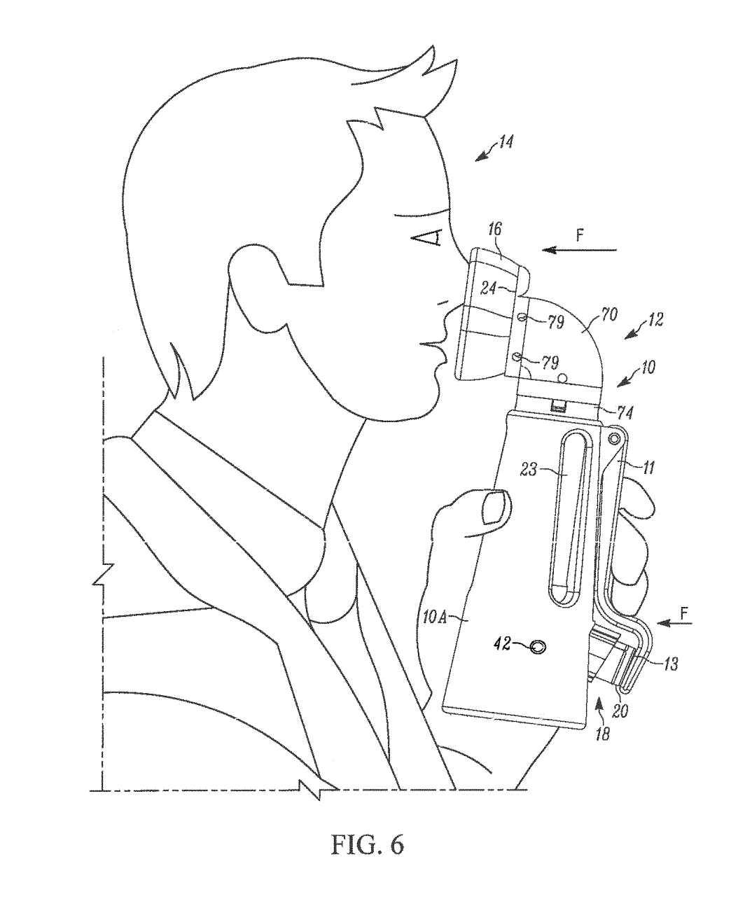

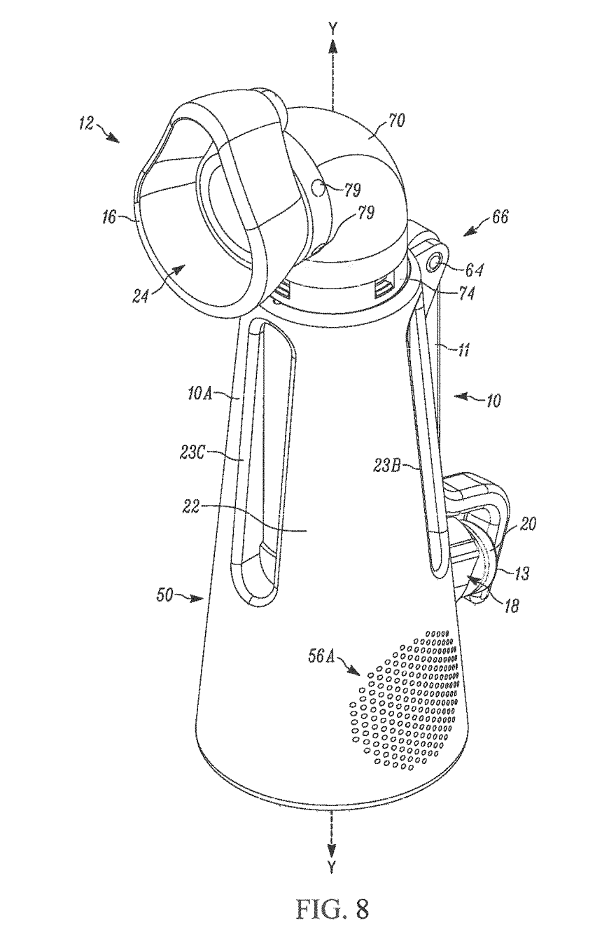

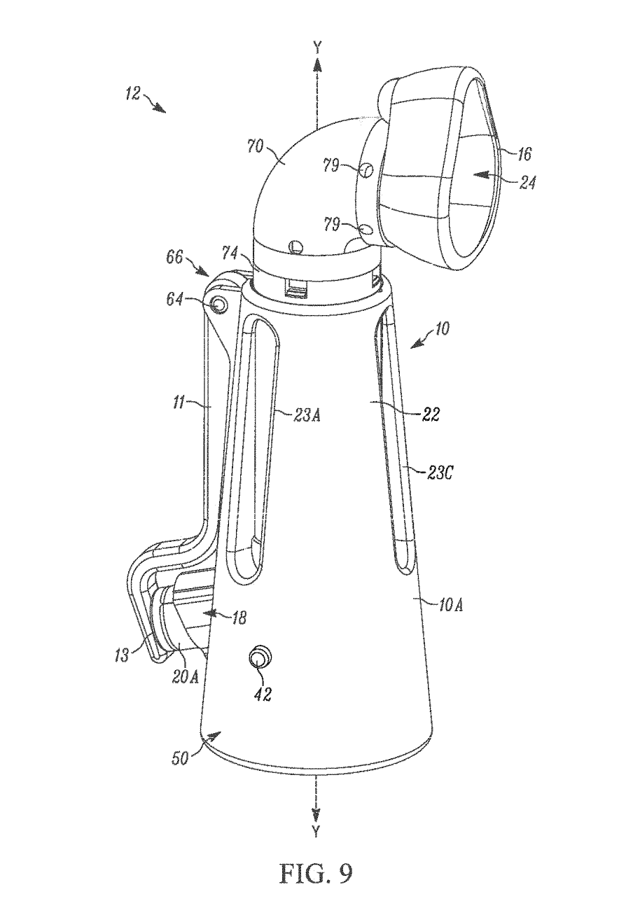

FIGS. 1-3, 6, 8-9, 29 and 33 illustrate an interactive spacer 10 for a respiratory device 12 in accordance with a first example embodiment of the present disclosure. The device 12 includes, in addition to the spacer 10, a mask 16 spaced apart by the space from a medication dispenser 18. The medication dispenser IS emits medication upon displacement of a button 20. The medication travels through a serially located passage 22 in a spacer body 10A of the spacer 10 out an opening 24 in the mask 16 for inhalation by a patient 14, as illustrated in FIGS. 1 and 6. In one example embodiment, the button 20 is displaced via a lever 11 of the spacer 10. The lever 11 is coupled to the spacer body 10A and interacts with the button 20 at contact point 13.

As in the illustrated example embodiment of FIG. 1A, a helper 14A is holding the patient 14 with one arm while holding the respiratory device 12 with the other. The helper 14A is holding the mask 16 to the patient's face, and by squeezing the lever 11 will administer the medication to the patient 14. As can be appreciated in FIG. 1A, the direction the lever 11 is displaced during the squeeze facilitates both the holding of the patient and the securing of the mask 16 to the patient's face.

In the illustrated example embodiments, the spacer body 10A comprises one or more lateral openings 23. In an example embodiment, the lateral openings 23 allow for easier assembly of the device 12, providing both access and visibility through the openings, acting as a viewing port to the internal area of the spacer body 10A. In an example embodiment, the spacer body 10A comprises a polymer, silicon, plastic, and/or metal. In an example embodiment, the spacer body 10A is formed utilizing 3D printing, molding, injection molding, transfer molding, etc.

In an alternative example embodiment, the spacer 10 is axially and horizontally located (not shown) and the medication from the dispenser 18 is emitted directly into the opening 24 of the mask 16 without passing through the spacer.

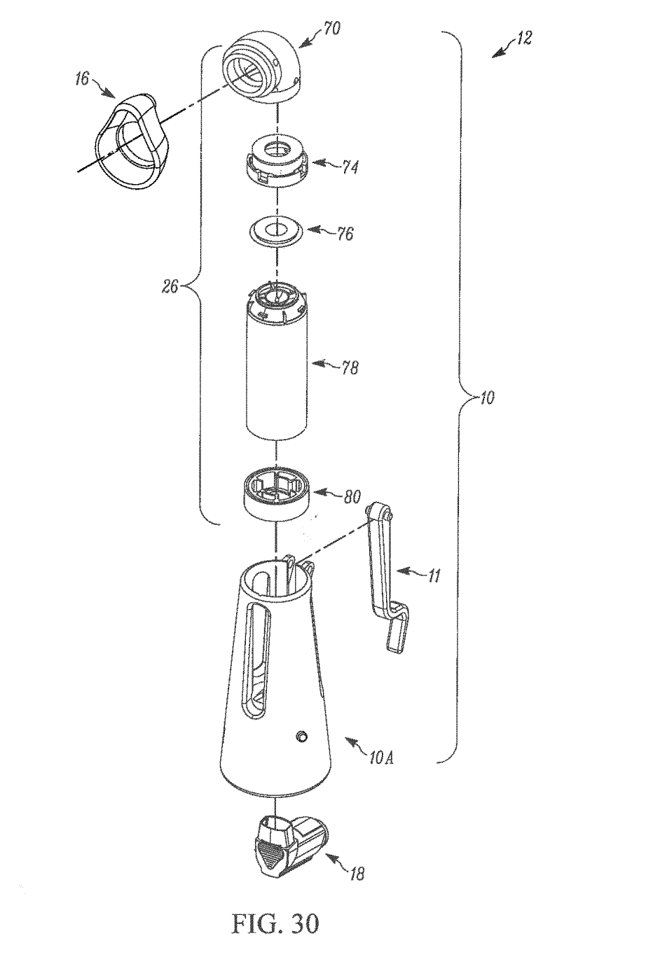

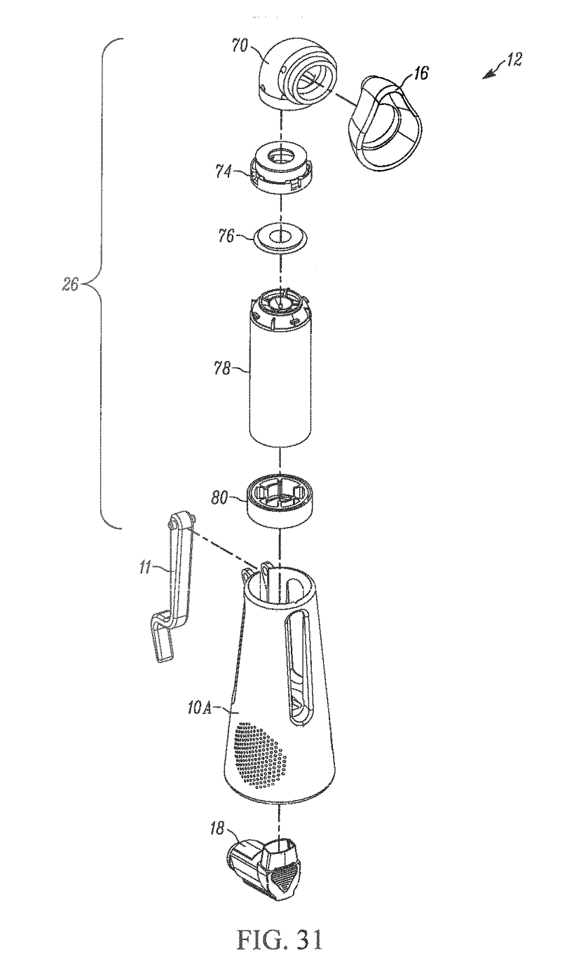

In the example embodiments of FIGS. 30-31, the spacer 10 is configured to attach to both the mask 16 and medication dispenser 18 through a sealing type connection 26. Such sealing type connection 26 can include, for example, threads, gaskets, press-fit seals, or any combination thereof. In the illustrated example embodiment, the sealing type connection 26 comprises at least one of an elbow 70, a spacer and cap 74, a spacer gasket 76, a spacer inner body 78, and a bottom cap 80. In another example embodiment, the components of the scaling type connection 26 are coupled together to form a fluid connection linking the mask to the medication dispenser 18.

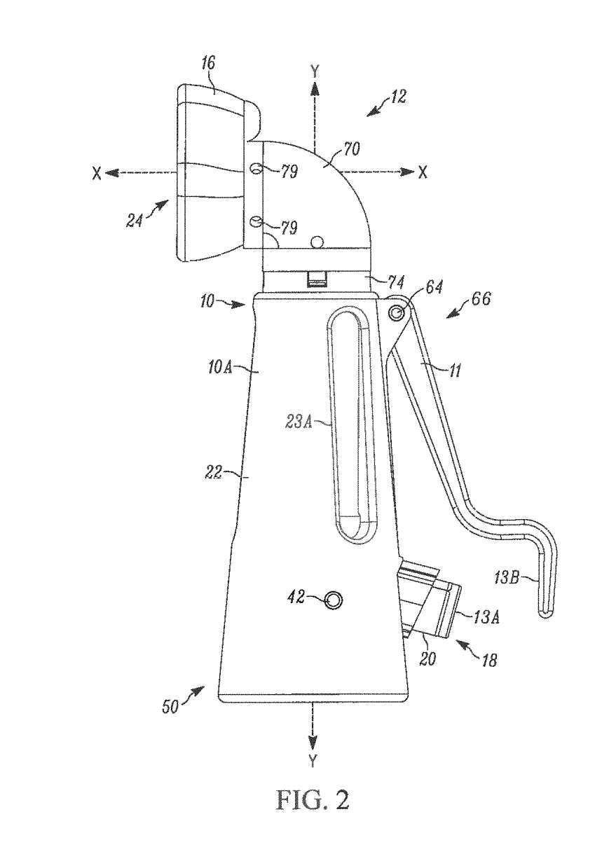

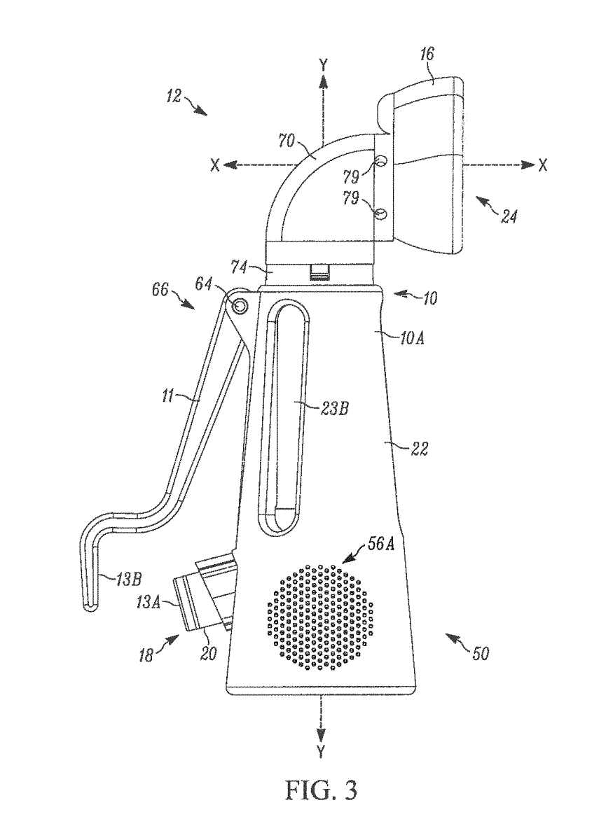

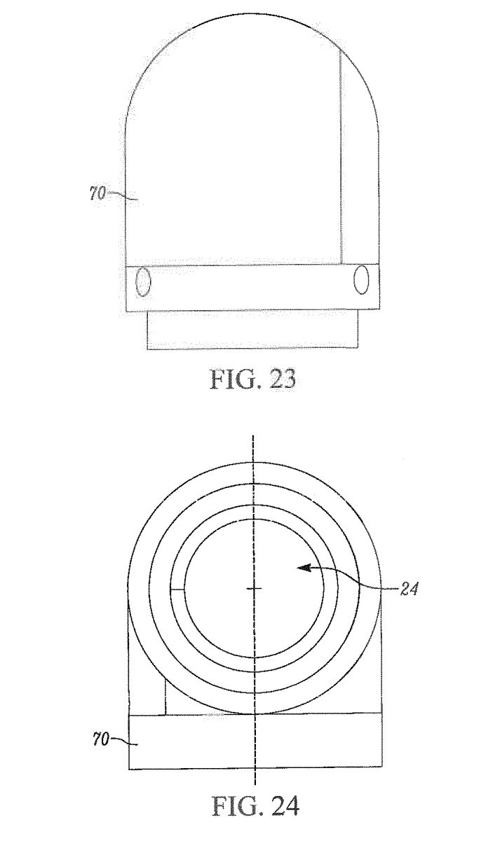

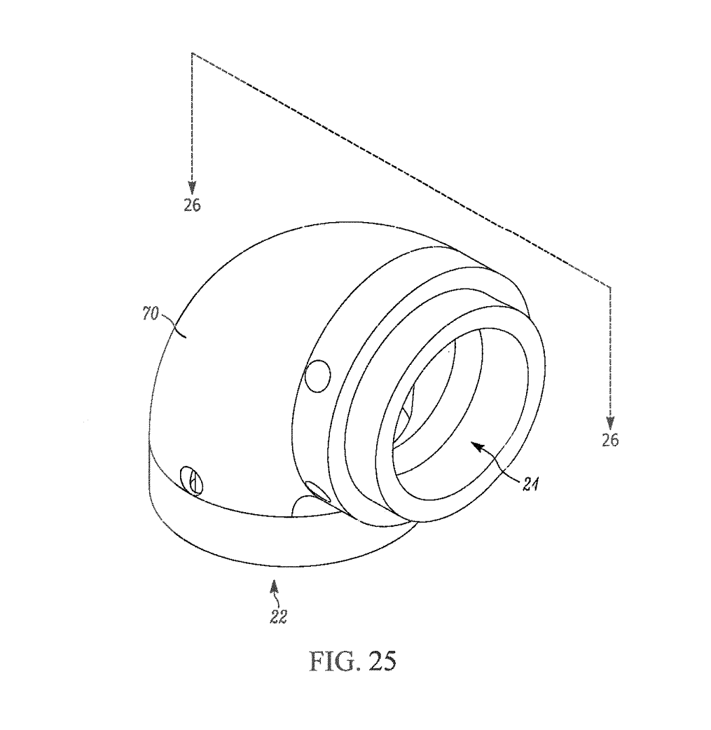

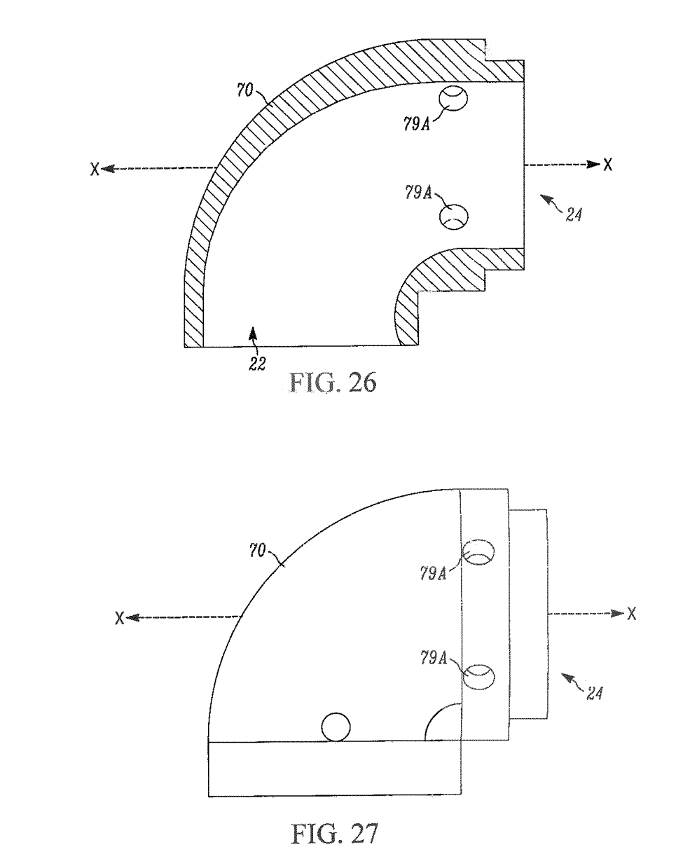

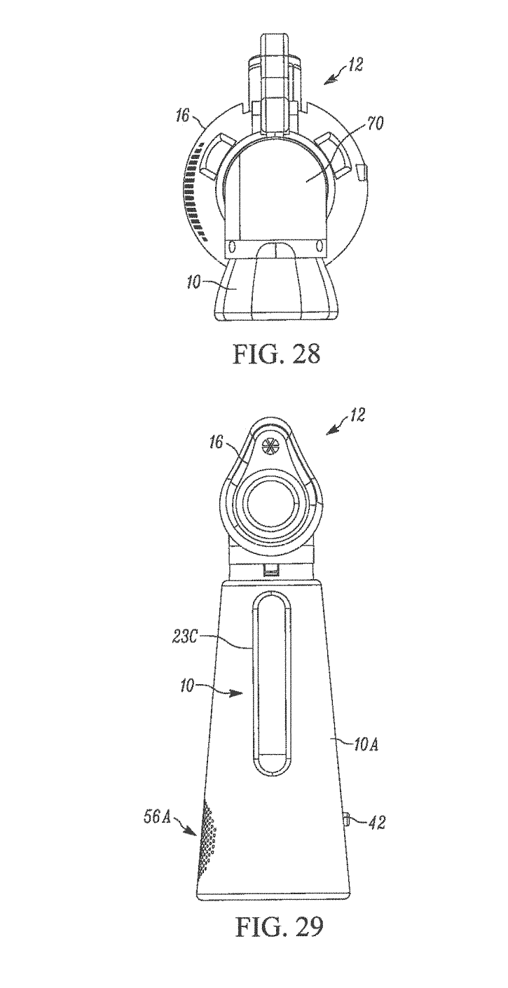

FIG. 2 is a left side elevation view of the interactive spacer device 10 in a relaxed position and FIG. 3 is a right side elevation view of the interactive spacer device in the relaxed position. The interactive spacer device 10 is coupled to the mask 16 and the medication dispenser 18 to form an assembly of the respiratory device 12. In one example embodiment, the interactive spacer device 10 comprises the elbow 70, wherein the elbow 70 couples the mask 16 to the spacer body 10A, as illustrated in FIGS. 23-27. The elbow 70 comprises a portion of the opening 24. In an example embodiment, the opening 24 is air tight, and fluidly links the mask 16 to the spacer 10. In the illustrated example embodiment of FIGS. 2 and 3, the elbow 70 is coupled to the spacer body 10A by the spacer end cap 74. The interactive spacer 10 comprises the substantially central passage 22 for the flow of medication along a first axis “Y”. As best seen in the illustrated example embodiment of FIG. 26, an internal shape of the elbow 70 can be altered relative to the external shape of the elbow, such that an angle of a turn that the medication takes to go from the serially located passage 22 to the opening 24 is greater than the external shape of the elbow would indicate. In the illustrated example embodiment, the elbow 70 comprises the same or similar material as the spacer body 10A and is formed in a same and/or similar manner. The mask 16 includes the opening 24 in fluid communication with the passage 22 along a second axis “X” transverse to the first axis Y. In one example embodiment, the opening 24 of the mask 16 is centrally located along the second axis X.

As illustrated in FIGS. 2-3, the button 20 is activated by compressing the lever 11 toward the spacer body 10A along an axis parallel to the second axis X. In an example embodiment, the lever 11, when compressed, is In an engaged position, as illustrated in FIGS. 1 and 6, and when the lever is not compressed, as illustrated in FIGS. 2-3, the lever is in the relaxed position. The lever 11 comprises a point of contact 13B chat interacts with a second point of contact 13A located on the medication dispenser 18. In an example embodiment, the second point of contact 13A is located on the button 20.

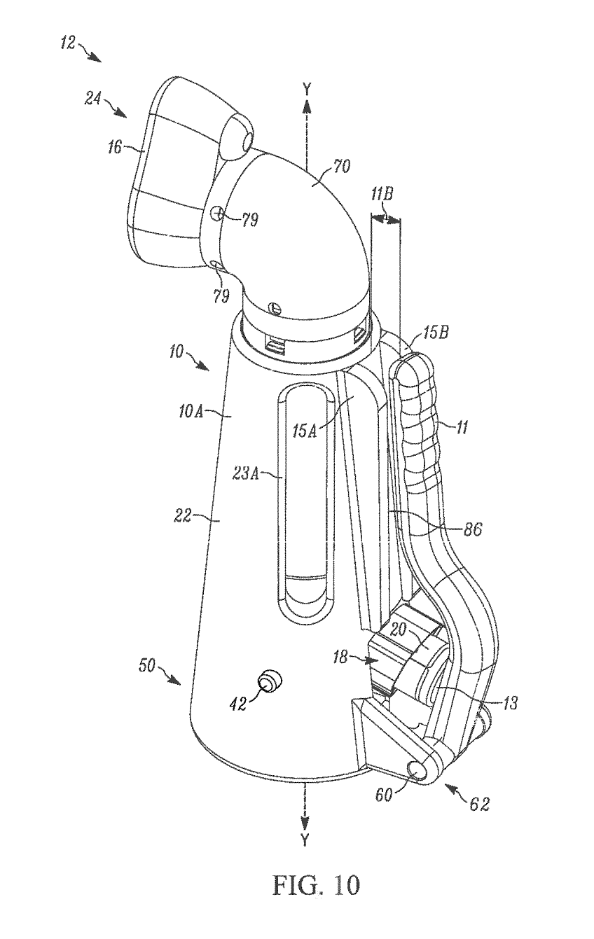

In the illustrated example embodiment of FIGS. 2-3, the lever 11 is coupled to the spacer body 10A via an upper hinge 66 comprising an upper pivot point 64. The lever 11 moves toward and away from the spacer body 10A via the upper pivot point 64. The spacer 10, including the lever 11, can be grasped by a single hand, such that the patient 14 or helper 14A can compress the lever while holding the spacer using the single hand, while, for example, holding the patient, as illustrated in FIG. 1A. In an example embodiment, the lever 11 can comprise indentations on an outer surface of the lever configured to conform to a general finger shape, such as illustrated In FIGS. 10-13.

In the illustrated example embodiments, and as best seen in FIG. 6, the spacer 10 separates the medication dispenser 18 from the mask 16 and aligns the forces, such that the force used to compress the lever 11 complements or assists n a separate or the same force applied by the patient 14 for sealing the mask 16 to the patient's face. Responsive the force F being above a force threshold, a sufficient seal is created between the mask 16 and the patient's face. In an example embodiment, the sufficient seal comprises a seal that prevents the passage of fluid or gas between the seal and the patients face. In another example embodiment, the sufficient seal comprises a seal that directs the medication from the medication dispenser 18 to the oropharynx of the patient 14 and does not allow the medication to escape between the mask 16 and the skin of the patient. Such orientation of the spacer 10 relative to the mask 16 and medication dispenser 18 reduces the proximity of the medication dispenser 18 to the patient's fate.

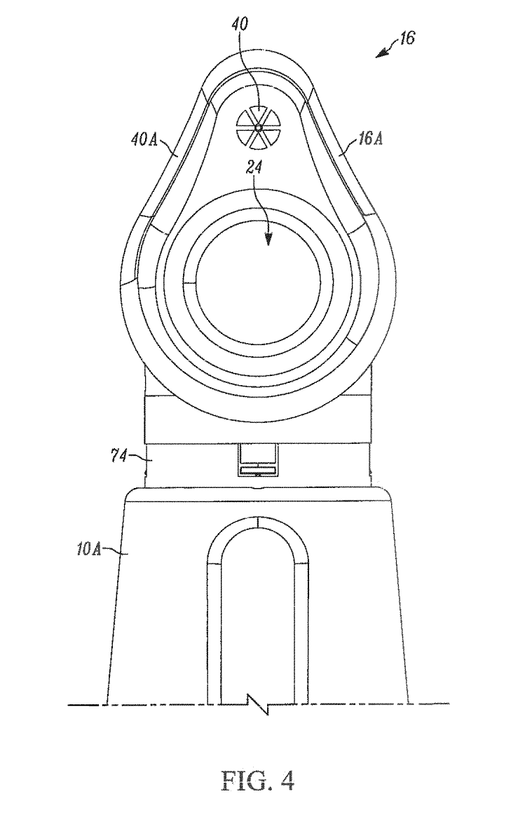

Illustrated in FIG. 4 is the mask 16, including a first switch 40 that is enabled when a sufficient seal is obtained between the mask and the patient 14. In an example embodiment, the first switch 40 comprises a proximity detector. In another example embodiment, the first switch 40 can be enabled responsive to threshold proximity of an object being established relative to the first switch. The threshold proximity comprises between 0.03 mm to about 5 mm. In an example embodiment, the first switch 40 comprises a pushbutton switch, as described with regard to the pushbutton switches comprising the recording, playback, and second switches 42B, 42, 86. In another example embodiment, the first switch can be in electrical communication with a sensing film 40A, that enables the first switch 40 responsive to threshold contact level, as programmed into a controller 54, being reached. In an example embodiment, the controller 54 is internal to the spacer body 10A as described below. The threshold contact comprises at least one of over 50%, 70%, and 90% of the sensing film 40A being in contact with the patient 14. In an example embodiment, the mask 16 is configured to simultaneously fit over a nose and mouth of the patient 14. In one example embodiment, the mask 16 comprises a sealing portion 16A that when in contact with the patient's face, and when sufficient force is applied, forms the seal. In another embodiment, the mask 16 comprises one or more capacitive, touch photosensitive, and/or resistive sensors (not shown), wherein the capacitive, touch photosensitive, and/or resistive sensors detect the presence of the patients face on the mask to identify the threshold contact level. In yet another embodiment, the mask 16 comprises multiple pushbutton switches around the sealing portion 16A, wherein a sufficient number of the multiple pushbutton switches are depressed, a sufficient seal is deemed to have been formed. It would be understood by one of ordinary skill in the art that the mask 16 can be sized for various patient sizes, as well as for exclusive nasal and oral inhalation. In the illustrated example embodiment, the mask 16 can comprise a silicon mask, one such silicon mask is a component in AeroChamber Plus® Flow-Vu® manufactured by Allergan®.

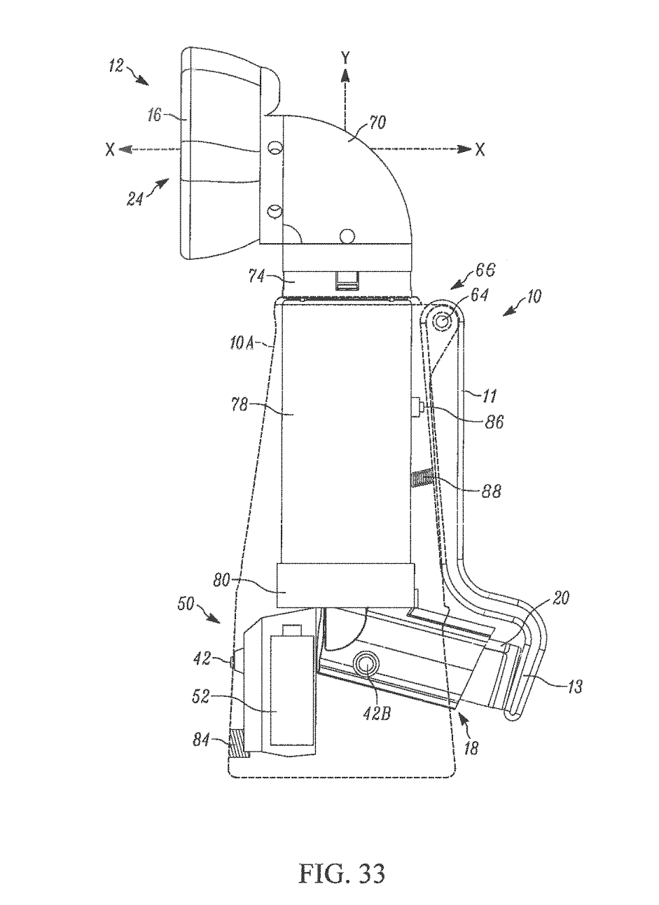

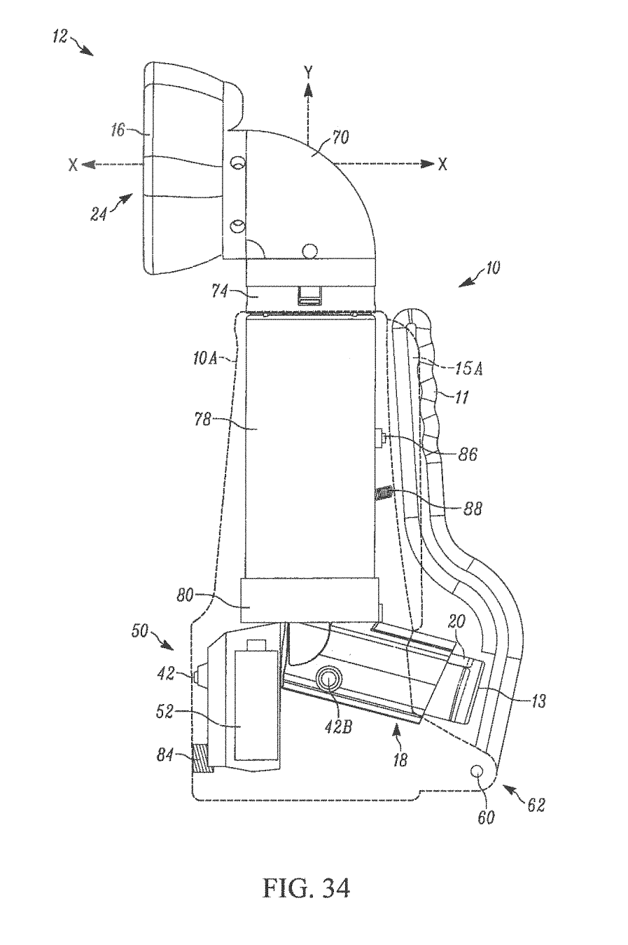

In an example embodiment, electronic circuitry 50 is located within the spacer 10 and functionally connected to first, recording, playback, and/or second switches 40, 42B, 42, 86, as best seen in FIGS. 4, 5, and 33-34. In an example embodiment, the electronic circuitry 50 enables the first, recording, playback, and/or second switch 40, 42B, 42, 86 responsive to an enabling signal from the first switch and/or depression of the recording, playback and/or second switch. In the illustrated example of FIGS. 33-34, the second switch 86 protrudes from the spacer 10 toward the lever 11, such that responsive to the lever being depressed, the second switch is likewise depressed by an inner surface of the lever.

Illustrated in FIGS. 1-2, 6, 9, and 33 is the playback switch 42 that is enabled when depressed by an operator. In one example embodiment, the actuation of the playback switch 42 activates a first playback, wherein the first playback may be a same or different playback that is activated responsive to the actuation of the second switch 86. In another example embodiment, the playback switch 42 is actuated by a parent to distract or amuse a child prior to dispensing the medication. While in one example embodiment, responsive to the playback switch 42 being actuated, the actuation of the second switch 86 will not activate a playback. In yet another example, the actuation of the second switch 86 will alter the playback from the playback activated by the playback switch 42, to a second playback.

In the illustrated example embodiment of FIG. 33, the recording switch 42B is functionally connected to a microphone 84 and a recording apparatus, such as an audio recorder. An appropriate audio recorder, microphone 84, and/or speaker 56 are included in the controller 54, one such controller is RadioShack Digital Recoding Module™, having part number: 276-1323. Further in the illustrated example embodiment, the controller 54 includes memory such as flash memory, random access memory, and/or road-only memory. In another example embodiment, the recording switch 42B does not protrude above an outer surface of the spacer body 10A, to prevent accidental recording during use. In yet another example embodiment, the recording switch 42B is located inside the spacer body 10A to prevent accidental recording during use. Responsive to depressing the recording switch 42B, one or more audio recordings of music and/or voice recordings can be recorded and stored in the memory of the controller 54 for playback, wherein playback of the one or more of the audio recordings is activated responsive to depression of the second switch 86 and/or the playback switch 42, as illustrated in FIG. 33. Though the playback switch 42 is depicted on a left side of the spacer 10, one of ordinary skill in the art would appreciate that the playback switch 42 could be located anywhere on the spacer.

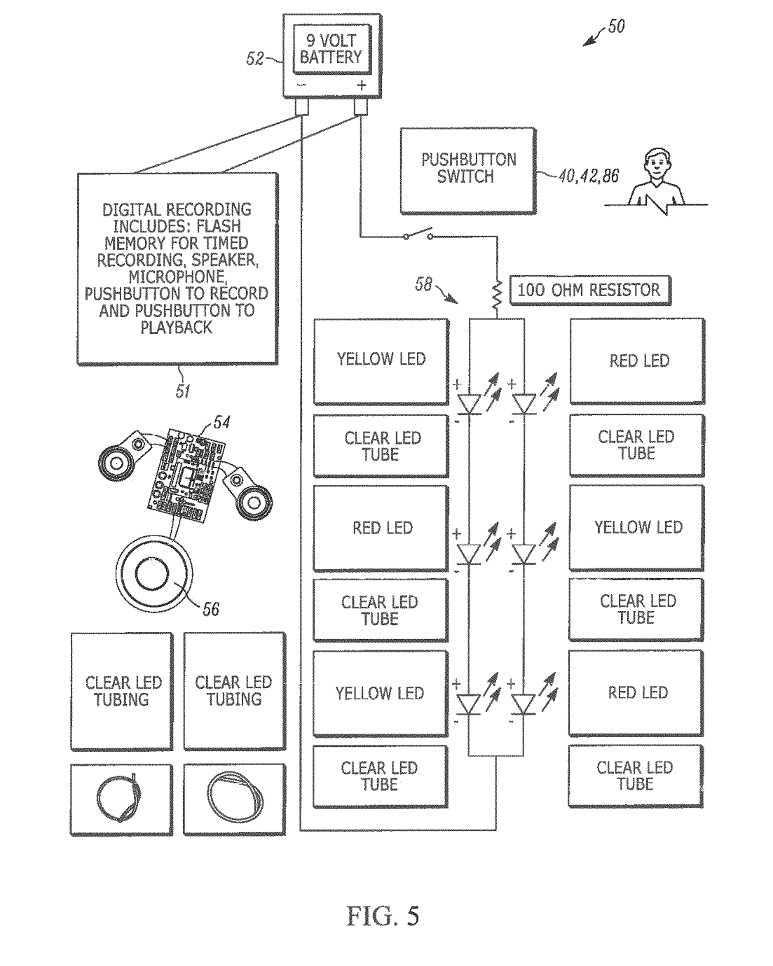

FIG. 5 is a schematic illustrating the electronic circuitry 50 of the interactive spacer 10 for the respiratory device 12 in accordance with another example embodiment of the present disclosure. The electronic circuitry 50 in the illustrated example embodiment is positioned on the spacer 10, the mask 16, and the medication dispenser 18. The electronic circuitry 50 includes a power supply 52, the controller 54, the first, recording, playback, and second switches 40, 42B, 42, 86 respectively, the speaker 56, and an illumination arrangement 58. In the illustrated example embodiment, the controller 54 includes the audio recorder, the speaker 56, and the microphone 84. In an example embodiment, the power supply 52 comprises a battery, one such battery would be a 9 volt battery that provides power to the controller 54 and supporting components.

In one example embodiment, the recording switch 42B comprises a pushbutton for recording sounds. In another example embodiment, the playback switch and/or the second switch 86 comprise pushbuttons for playing back sounds, such as messages and/or musical tones. In the illustrated example embodiment, the recording, the playback, and/or second switches 42B, 42, 86 comprise the pushbutton, one such suitable example of a pushbutton would be SparkFun Mini pushbutton Switch™ manufactured by SparkFun Electronics™ and having a stock keeping unit number of COM-00097. In one example embodiment, a button extension is placed over the second switch 86 to make the second switch easy to engage.

Further, in the example embodiment, the illumination arrangement 58 comprises a series of light emitting diodes that light up when one or more of the first, recording, playback, and/or second switches 40, 428, 42, 86 at enabled. One suitable example of an LED would be SparkFun Super Bright Yellow LED™ manufactured by SparkFun Electronics™ and having a stock keeping unit number of COM-00530. In one example embodiment, the light emitting diodes (LEDs) light up responsive to the recording switch 42B being depressed to indicate that the audio recorder is recording. In another example embodiment, the LEDs light up responsive to the second switch 86 being depressed to indicate that the medication dispenser 18 is dispensing medication. In yet another example embodiment, the illumination arrangement 58 comprises multiple illumination colors, and the controller 50 can instruct the illumination arrangement 58 to emit a particular color of light for a particular switch being activated (e.g., red for the playback switch 42, blue for the second switch 86, etc.).

In the illustrated example embodiment of FIG. 5, the controller 54 allows for a voice message or music to be recorded by the audio recorder for a duration of time and played back to assist and/or motivate the user of the device 12. The voice message and/or music is enabled in one example embodiment with the activation of the first, playback, rd/or second switch 40, 42, 86. In addition, the controller 54 further allows, with the enablement of the first, playback, and/or second switch 40, 42, 86, a combination of lights from the illumination arrangement 58 to be emitted with the message and/or music from the speaker 56.

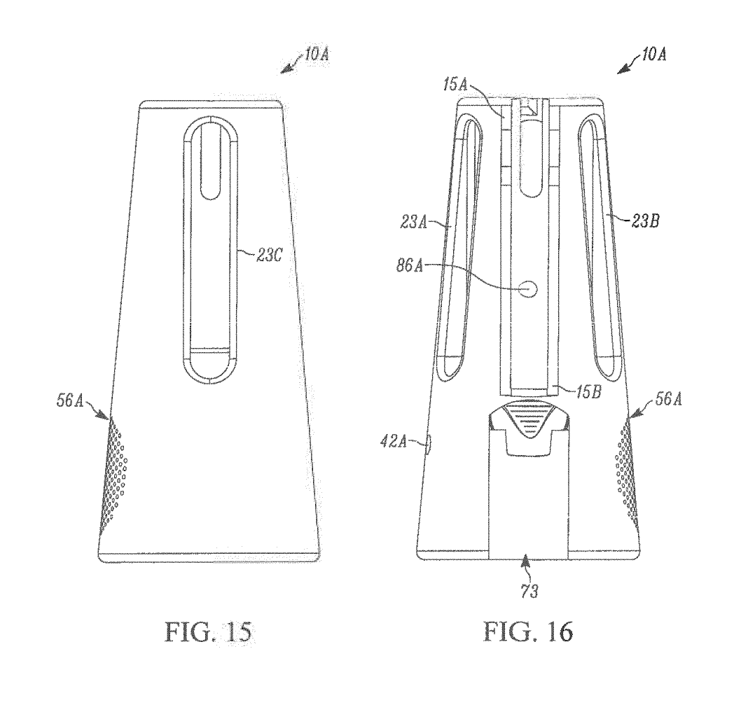

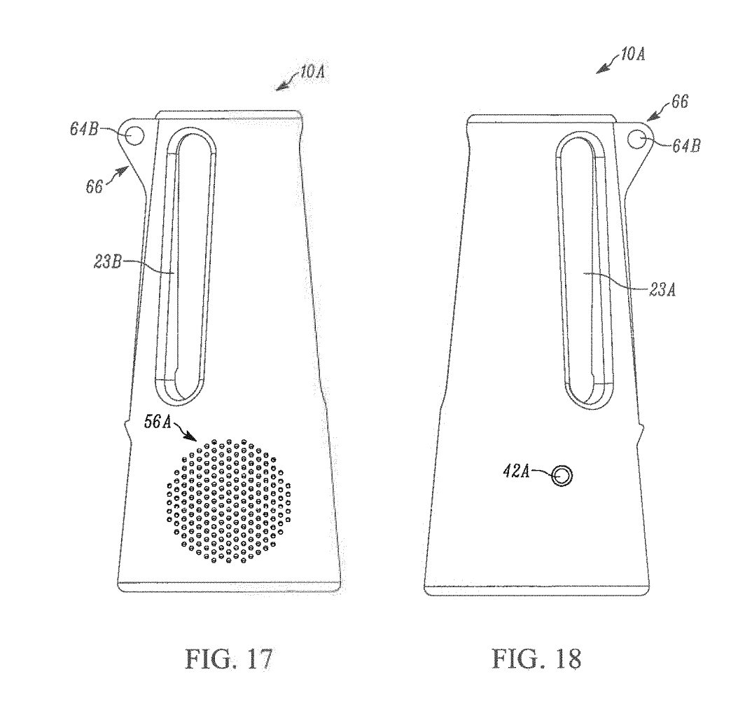

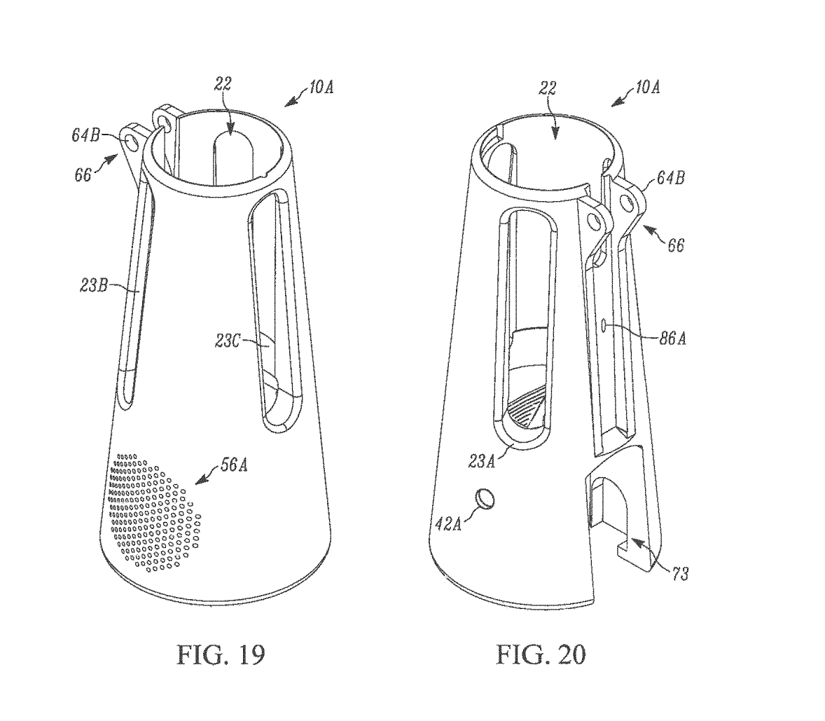

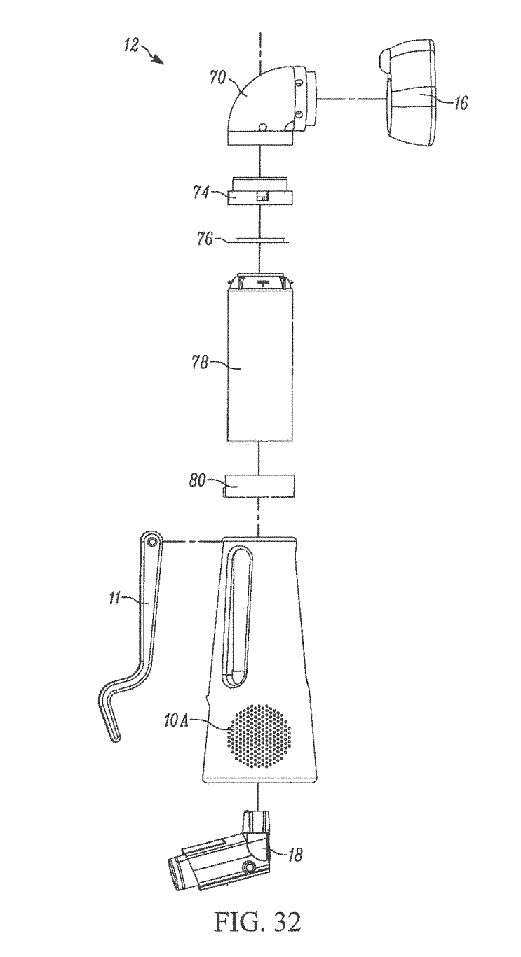

In the illustrated example embodiment of FIGS. 3, 8, 15-17, 19, 29, and 31-32, the spacer body 10A comprises audio openings 56A to facilitate the audio emission of the speaker 56. In the illustrated example embodiment, the illumination arrangement 58 emits light through a plurality of illumination openings 79 located on the elbow 70. In one example embodiment, responsive to depression of the button 20 and/or the second switch 86, the illumination arrangement 58 emits light through the lateral openings 23A-23C spaced around the spacer body 10A.

Referring now again to the example embodiment of FIGS. 1A, 1 and 6, the patient 14 or the helper 14A is administering medication from the device 12 by placing the mouth piece 16 over the patient's nose and mouth with a force F. The force F is generated by the advancement of the patient's or helper's hand to depress the lever 11 into interacting with the medication dispenser 18 at the contact point 13. While the force F is being applied, the patient 14 or the helper 14A displaces the first switch 40 when proper contact is achieved with the mask 16 and substantially simultaneously engages the second switch 86 via the lever 11. The engagement of the switches, 40, 86, provides a signal by either sound and/or lights to the patient 14 from the controller 54 and the circuitry 50 to train or encourage the patient using the device 12. In yet another embodiment, the button 20 and the second switch 86 are combined to perform the collective functions of releasing medication and to operate sound/lights in combination with or without the second switch 86. That is, the button 20 and second switch 86 are combined as a single switch when enabled to provide I/O to the controller 54, the controller operating both the sound and release of medication. In an example embodiment, the single switch is located on the point of contact 13B on the lever 11.

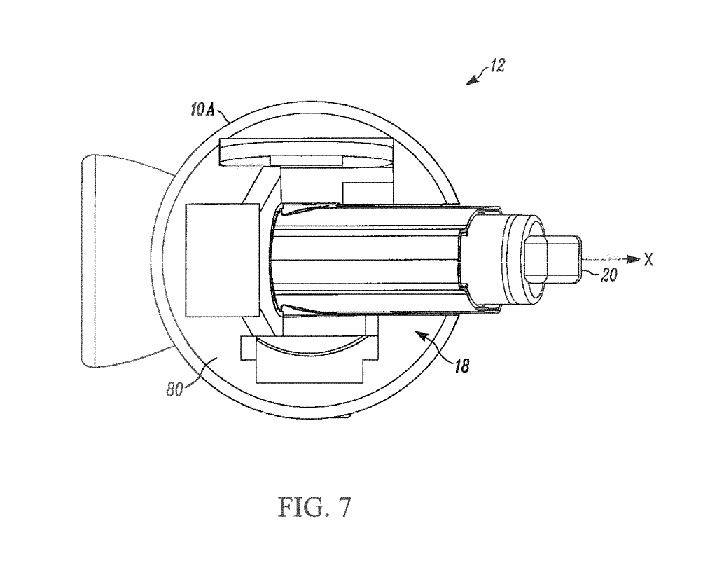

The spacer 10, in the example embodiment of FIGS. 1-3, 6, and 8-9, supports much of the circuitry 50 and the controller 54 and advantageously moves the force eccentrically away from the mouth piece 16, allowing the patient 14 a forward view. That is, the device 12 is outside of the patient's line-of-eight, while the patient 14 is depressing the lever 1 to receive the medication through the mask 16. Additionally, the local power supply 52 allows the device 12 to be completely portable for the patient 14. Further, the medication dispenser 18 is easily integrated into the spacer 10. As illustrated In FIG. 7, the medication dispenser 18 is coupled to the bottom of the spacer body 10A via the bottom cap 80. In one example embodiment, the bottom cap 80 comprises a standard spacer silicon cap that can be molded and/or formed to couple to one or more differently sized medication dispensers.

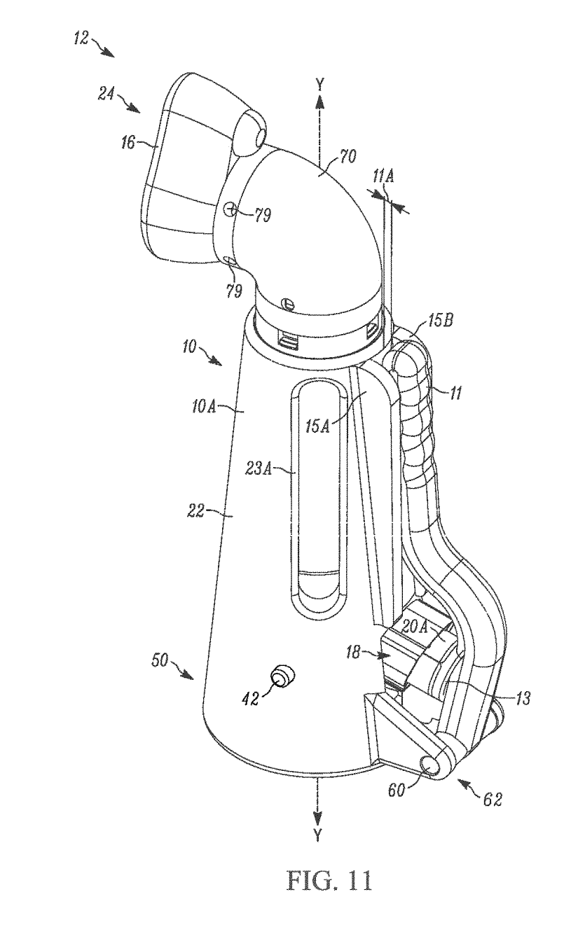

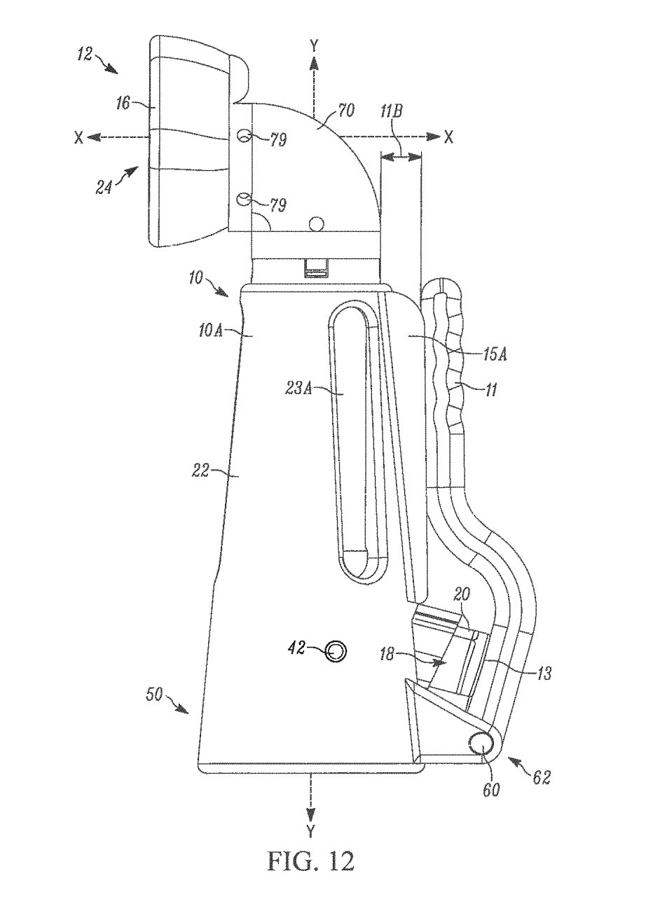

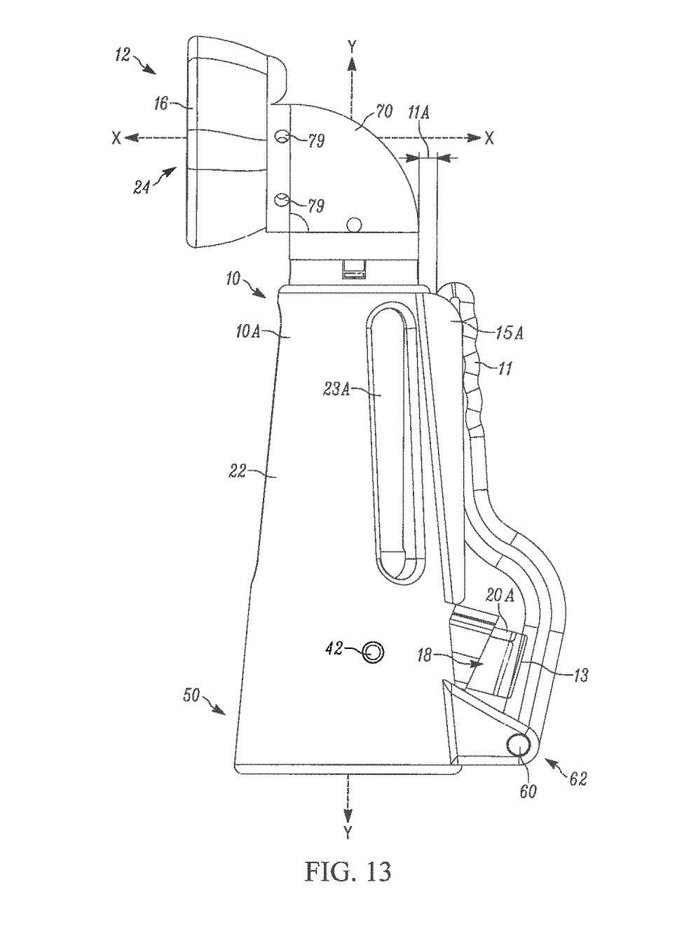

Turning to a second embodiment, illustrated In FIGS. 1A, 10-13, and 34, the spacer 10 is different from the first embodiment in that the lever 11 is coupled to the spacer body 10A via a bottom hinge 62 comprising a bottom pivot point 60. In the illustrated example embodiment, the lever 11, when depressed, fits between two lateral protrusion 15A, 15B that protrude from the spacer body 10A. The lateral protrusions 15A-15B prevent the patient 14 from accidently activating the second switch 86 absent depression of the lever 11. Responsive to being depressed, the lever 11 depresses the second switch 86 to activate the lights and/or the playback, in conjunction with the first switch 40 being enabled. In the illustrated example embodiment, the lever 1 will interact with a spring 88 that will express the lever away from the spacer body 10A absent the depressive force. The spring 88 prevents the device 12 from dispensing the medication absent the depressive force. The bottom pivot point 60 decreases an amount of force required to depress the lever 11 proportionally to the length of the lever. In the second example embodiment, the lever 11 rotates about the axis created by the bottom pivot point 60, such that the button 20 is depressed using torque provided by the lever, rather than direct force. In the illustrated example embodiment of FIGS. 11 and 13, the lever 11 is in the engaged position, wherein the lever is a first distance 11A away from the spacer body 10A. The lever 11 is depressing the button 20 and the second switch 86 when in the engaged position. In the illustrated example embodiment of FIGS. 10 and 12, the lever 11 is in the relaxed position, wherein the lever is a second distance 11B away from the spacer body 10A. The lever 11 is not depressing the button 20 and the second switch 86 when in the relaxed position. The first distance 11A is less than the second distance 11B.

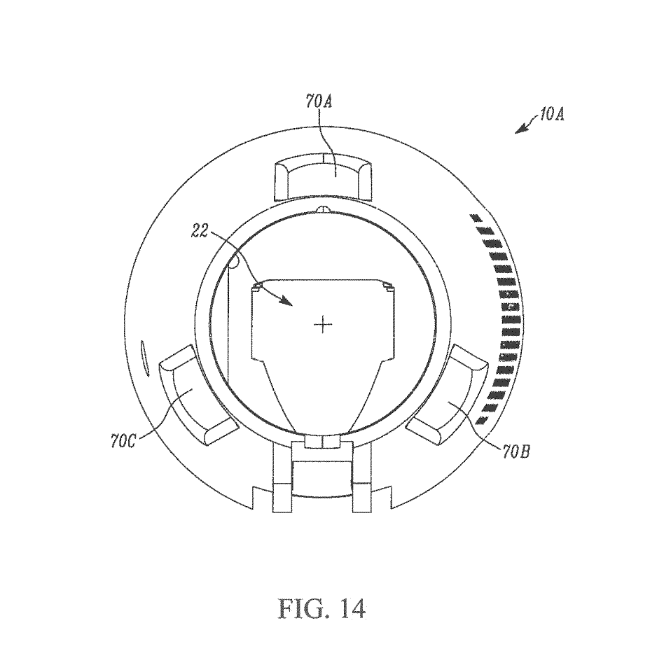

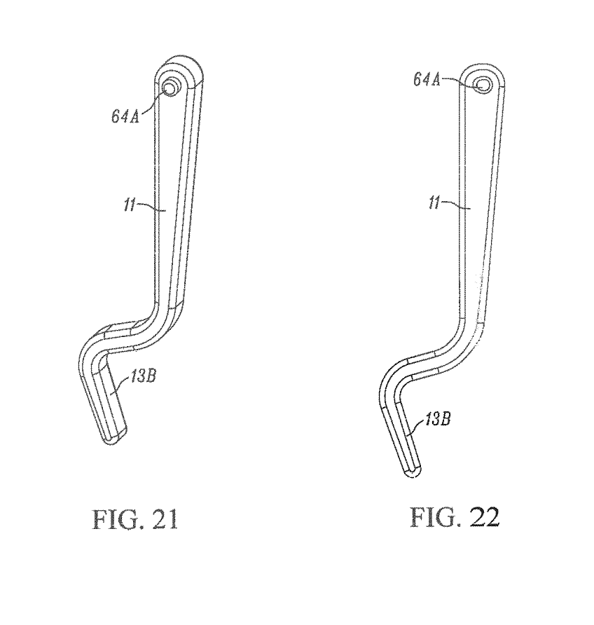

As illustrated in example embodiment of FIG. 14, the spacer body 10A comprises a plurality of locking protrusions 70A, 70B, and 70C. The locking protrusions 70A, 70B, and 70C interact with the at least one of the elbow 70, the spacer end cap 74, and the spacer gasket 76 to couple the elbow to the spacer body 10A. The spacer body 10A further comprises a medication dispenser opening 73, as illustrated in FIGS. 16 and 20. The medication dispenser opening 73 is configured to fit and secure the medication dispenser 18 in a position, in conjunction with the bottom cap 80, such that the button 20 is aligned with the point of contact 13B on the lever 11. In the illustrated example embodiment of FIGS. 16, 18, and 20, the spacer body 10A comprises a playback switch opening 42A and a second switch opening 86A. In an example embodiment, the playback switch opening 42A and the second switch opening 86A are configured to encircle the playback and second switches 42, 86, respectively. The spacer body 10A further comprises upper pivot point openings 64B that, in conjunction with a lever pivot point opening 64A, illustrated in FIGS. 21 and 22, and the pivot point 64, form the upper pivot joint 66. In one example embodiment, the upper pivot point 64 comprises a shaft extending through the lever pivot point openings 64A and extending beyond the upper pivot point openings 64B. The upper pivot point 64 further comprises ends having a width greater than the upper pivot point openings 64A to couple the lever 11 to the spacer body 10A. The upper pivot point 64 is formed in substantially the same manner as the lower pivot point 60, with equivalent components titled lower rather than upper.

In the illustrated example embodiment of FIGS. 30-33, the spacer 10 comprises one or more components. The spacer components include the elbow 70, the spacer end cap 74, the spacer gasket 76, the spacer inner body 78, the bottom cap 80, the spacer body 10A and the lever 11. In an example embodiment, the spacer end cap 74 comprises a silicon end cap, one such suitable example silicon end cap is a component in AeroChamber Plus® Flow-Vu® manufactured by Allergan®. In an example embodiment, the spacer gasket 76 can comprise a unidirectional air flow gasket, one such suitable example gasket is a component in AeroChamber Plus® Flow-Vu® manufactured by Allergan®. In an example embodiment, the spacer inner body 78 can comprise an airtight container, one such suitable example container is a component is AeroChamber Plus® Flow-Vu® manufactured by Allergan®

In an example embodiment, the spacer components of the spacer 10 comprise at least one of a polymer, silicon, plastic, metal, or the like. The spacer components of the spacer 10 can have a same or different material relative to each other. In one example embodiment, the spacer components are formed in a same or similar manner as described above with regard to the spacer body 10A. The spacer components of the spacer 10 can be formed in a same or different manner relative to each other. In another example embodiment, the components of the spacer 10, when assembled, form the serially located passage 22 to comprise a smooth inner surface for air tight and/or laminar flow of the medication from the medication dispenser 18 to the opening 24 of the mask 16. The spacer 10 increases an ease with which medication is taken and helps to train patients 14 how to properly take the medication utilizing the playback and/or illumination.

In the foregoing specification, specific embodiments have been described. However, one of ordinary skill in the art appreciates that various modifications and changes can be made without departing from the scope of the disclosure as set forth in the claims below. Accordingly, the specification and figures are to be regarded in an illustrative rather than a restrictive sense, and all such modifications ma intended to be included within the scope of present teachings.

The benefits, advantages, solutions to problems, and any element(s) that may cause any benefit, advantage, or solution to occur or become more pronounced are not to be construed as a critical, required, or essential features or elements of any or all the claims. The disclosure is defined solely by the appended claims including any amendments made during the pendency of this application and all equivalents of those claims as issued.

Moreover in this document, relational terms such as first and second, top and bottom, and the like may be used solely to distinguish one entity or action from another entity or action without necessarily requiring or implying any actual such relationship or order between such entities or actions. The terms “comprises,” “comprising” “has”, “having,” “includes”, “including” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof are defined as being close to as understood by one of ordinary skill in the art. In one non-limiting embodiment the terms are defined to be within for example 10% in another possible embodiment within 5%, in another possible embodiment within 1%, and in another possible embodiment within 0.5%. The term “coupled” as used herein is defined as connected or in contact either temporarily or permanently, although not necessarily directly and not necessarily mechanically. A device or structure that is “configured” in a certain way is configured in at least that way, but may also be configured in ways that are not listed.

To the extent that the materials for any of the foregoing embodiments or components thereof are not specified, it is to be appreciated that suitable materials would be known by one of ordinary skill in the art for the intended purposes.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separately claimed subject matter.

What is claimed is:

1. A respirator device for dispensing medication, the device comprising:
a mask for covering at least one of a mouth and a nose of a patient;
a medication dispenser for housing medication to be received by the patient upon activation of a button by a lever, said lever coupled to a spacer body of an interactive spacer, the button located on the dispenser; and
the interactive spacer eccentrically located and spacing said mask from said medication dispenser, said spacer comprising a power supply and interactive controller for providing at least one of sound and light upon activation of a first switch located on aid device.

2. The respiratory device of claim 1 wherein said first switch is located within said mask, the first switch being activated by contact of a patient's face upon making a sufficient seal with said mask during use.

3. The respiratory device of claim 1 further comprising a second switch, said second switch is positioned on said medication dispenser and said at least one of sound and light being activated upon enabling of both said first and said second switch.

4. The respiratory device of claim 3 wherein the operation of said button and second switch we combined into a single switch.

5. The respiratory device of claim 1 wherein said interactive spacer comprises a passage for said medication along a first axis and said mask includes an opening in fluid communication with said passage along a second axis transverse to said first axis.

6. The respiratory device of claim 5 wherein said opening of said mask is centrally located along said second axis, said button being activated by depressing the lever into contact with said button in a direction parallel with said second axis.

7. The respiratory device of claim 1 wherein activation of said first switch comprises the patient having a threshold level of contact with the mask as measured by a sensing film.

8. The respiratory device of claim 1 wherein enabling a recording switch, located on said device, activates a recording device to record the sound to be played upon activation of the first switch.

9. The respiratory device of claim 1 wherein said first switch comprises a proximity sensor, and wherein the activation of said first switch comprises the first switch detecting an object within a threshold proximity.

10. The respiratory device of claim 1 wherein the lever is coupled to the spacer body via an upper pivot joint, the upper pivot joint located between the mask and the medication dispenser.

11. The respiratory device of claim 1 wherein the lever is coupled to the space body via a lower pivot joint, the medication dispenser located between the lower pivot joint and the mask.

12. The respiratory device of claim 1 wherein the interactive spacer assembly comprises a substantially smooth and airtight central passage wherein the medication flows from the medication dispenser through the central passage to the mask.

13. The respiratory device of claim 1 wherein said interactive controller is in communication with a speaker and an illumination assembly.

14. The respiratory device of claim 1 wherein the spacer body comprises two lateral protrusions that protrude from the spacer body, wherein the lever, responsive to being depressed, is housed between the two lateral protrusions.

15. The respiratory device of claim 14 wherein a second switch is located between the two lateral protrusions.

16. A method of dispensing medication from a respiratory device comprising:
activating a first switch by identifying that a mask is covering at least one of a nose and mouth of a patient, the mask coupled to a modification dispenser by an interactive spacer, the interactive spacer eccentrically located and spacing said mask from said medication dispenser, the first switch in electrical communication with a controller located in the interactive spacer;
identifying a depression of a lever to activate a button located on the dispense the lever coupled to a spacer body of the interactive spacer, the button in electrical communication with the controller; and
responsive to activating the first switch and identifying the depression of the lever:
dispensing the medication from the medication dispenser through the interactive spacer to the mask; and
providing at least one of sound and light as provided by the controller in electrical communication with at least one of a speaker and an illumination assembly.

17. The method of claim 16 wherein activating said first switch comprises identifying that the patient has a threshold level of contact with the mask.

18. The method of claim 16 wherein activating said first switch comprises detecting an object within a threshold proximity of the first switch, further wherein the threshold proximity is set by prescribed parameters in said controller.

19. The method of claim 16 comprising responsive to identifying an activation of a second switch and enablement of the first switch, said second switch positioned on said medication dispenser, activating said at least one of sound and light.

20. A respiratory device for dispensing medication, the device comprising:
a mask for covering at least one of a mouth and a nose of a patient;
a medication dispenser for housing medication to be received by the patient upon activation of a button by a lever, said lever coupled to a spacer body of an interactive spacer, the button located on the dispenser; and
the interactive spacer eccentrically located and spacing said mask from said medication dispenser, said interactive spacer comprising a passage for said medication along a first axis and said mask includes an opening in fluid communication with said passage along a second axis transverse to said first axis, and wherein said spacer comprises a power supply and interactive controller for providing at least one of sound and light upon activation of a first switch located within maid mask, the first switch being activated by contact of a patient's face upon making a sufficient seal with said mask during use.
